(19) United States
(12) Patent Application Publication
(10) Pub. No.: US 2009/0149930 A1
(43) Pub. Date: Jun. 11, 2009

(54) APPARATUS AND METHODS FOR COOLING A TREATMENT APPARATUS CONFIGURED TO NON-INVASIVELY DELIVER ELECTROMAGNETIC ENERGY TO A PATIENT'S TISSUE

(75) Inventor: Alan Schenck, Sunnyvale, CA (US)

Correspondence Address: WOOD, HERRON & EVANS, LLP, 2700 CAREW TOWER, 441 VINE STREET, CINCINNATI, OH, 45202, US

(73) Assignee: THERMAGE, INC., Hayward, CA (US)

(21) Appl. No.: 11/952,649

(22) Filed: Dec. 7, 2007

(51) Int. Cl. A61N 5/06

(52) U.S. Cl. 607/100

(57) ABSTRACT

Apparatus and methods for delivering electromagnetic energy to a patient's tissue. The treatment apparatus includes a closed-loop cooling system that cools the energy delivery device. The fluid forced to flow in the closed-loop cooling system is chilled to a first temperature at a location remote from the energy delivery device and is warmed to a higher second temperature near the energy delivery device. This promotes better control over the fluid temperature at the energy delivery device.

### FIELD OF THE INVENTION

The invention generally relates to apparatus and methods for treating tissue with electromagnetic energy and, more particularly, relates to apparatus and methods for cooling a treatment device used to deliver electromagnetic energy to a patient's tissue.

### BACKGROUND OF THE INVENTION

Energy delivery devices that can non-invasively treat tissue with electromagnetic energy are extensively used to treat a multitude of diverse skin conditions. Among other uses, non-invasive energy delivery devices may be used to tighten loose skin so that a patient appears younger, to remove skin spots or hair, or to kill bacteria. Such non-invasive energy delivery devices emit electromagnetic energy in different regions of the electromagnetic spectrum for tissue treatment.

High frequency treatment devices, such as radio-frequency (RF)-based devices, may be used to treat skin tissue non-ablatively and non-invasively by passing high frequency energy through a surface of the skin, while actively cooling the skin to prevent damage to the skin's epidermal layer. The high frequency energy heats tissue beneath the epidermis to a temperature sufficient to denature collagen, which causes the collagen to contract and shrink and, thereby, tighten the tissue. Treatment with high frequency energy also causes a mild inflammation. The inflammatory response of the tissue causes new collagen to be generated over time (between three days and six months following treatment), which results in further tissue contraction.

Typically, treatment devices include a treatment tip that is placed in contact with, or proximate to, the patient's skin surface and that emits electromagnetic energy that penetrates through the skin surface and into the tissue beneath the skin surface. The non-patient side of the energy delivery device, such as an electrode, in the treatment tip may be sprayed with a coolant or cryogen spray under feedback control of temperature sensors for cooling tissue at shallow depths beneath the skin surface. A controller triggers the coolant spray based upon an evaluation of the temperature readings from temperature sensors in the treatment tip.

The cryogen spray may be used to pre-cool superficial tissue before delivering the electromagnetic energy. When the electromagnetic energy is delivered, the superficial tissue that has been cooled is protected from thermal effects. The target tissue that has not been cooled or that has received nominal cooling will warm up to therapeutic temperatures resulting in the desired therapeutic effect. The amount or duration of pre-cooling can be used to select the depth of the protected zone of untreated superficial tissue. After the delivery of electromagnetic energy has concluded, the cryogen spray may also be employed to prevent or reduce heat originating from treated tissue from conducting upward and heating the more superficial tissue that was cooled before treatment with the electromagnetic energy.

Although conventional apparatus and methods for delivering cryogen sprays have proved adequate for their intended purpose, what is needed are improved apparatus and methods for cooling superficial tissue in conjunction with non-invasive treatment of deeper tissue with electromagnetic energy.

### SUMMARY OF THE INVENTION

In one embodiment, an apparatus is provided for treating tissue with electromagnetic energy. The apparatus comprises an energy delivery device configured to transfer the electromagnetic energy to the tissue. The energy delivery device includes a manifold body, a channel in the manifold body, an inlet to the channel, and an outlet from the channel. A closed-loop cooling system is coupled in a circulation loop with the inlet and the outlet of the channel. The closed-loop cooling system includes a pump configured to pump the fluid in the circulation loop to the inlet of the channel and through the channel to the outlet from the channel. A heat exchange member is disposed in the circulation loop between the pump and the inlet to the channel. The heat exchange member is configured to heat the fluid before the fluid enters the inlet of the channel.

In another embodiment, a method is provided for treating tissue with electromagnetic energy. The method comprises pumping the fluid at a first temperature from a reservoir to an energy delivery device and circulating the fluid at a second temperature through the energy delivery device. The fluid is heated at a location between the reservoir and the energy delivery device to the second temperature, which is greater than the first temperature. The method further comprises returning the fluid from the energy delivery device to the reservoir and delivering the electromagnetic energy from the energy delivery device to the tissue beneath the skin surface.

### BRIEF DESCRIPTION OF THE DRAWINGS

The accompanying drawings, which are incorporated in and constitute a part of this specification, illustrate embodiments of the invention and, together with a general description of the invention given above and the detailed description of the embodiments given below, serve to explain the principles of the invention.

FIG. 1 is a diagrammatic view of a treatment system with a handpiece, a treatment tip, and a console in accordance with an embodiment of the invention

FIG. 2 is a diagrammatic view of the handpiece, treatment tip, and console of FIG. 1 showing a closed-loop cooling system of the treatment system.

FIG. 3 is a rear view of the assembled treatment tip taken generally along line 3-3 in FIG. 2 showing the electrode and temperature sensors.

FIG. 4 is a perspective view of the handpiece partially shown in phantom and a heat exchanger of the closed-loop cooling system inside the handpiece in which certain internal components, such as electrical wiring, are omitted for clarity.

FIG. 5 is an exploded view of the treatment tip of FIG. 2 in which the treatment electrode is shown in an unfolded condition.

FIG. 6 is a front perspective view of a manifold body located inside the treatment tip of FIG. 5.

FIG. 7 is a rear perspective view of the manifold body of FIG. 6.

FIG. 8 is an exploded perspective view of the heat exchanger located in the handpiece of FIGS. 2 and 4.

### DETAILED DESCRIPTION

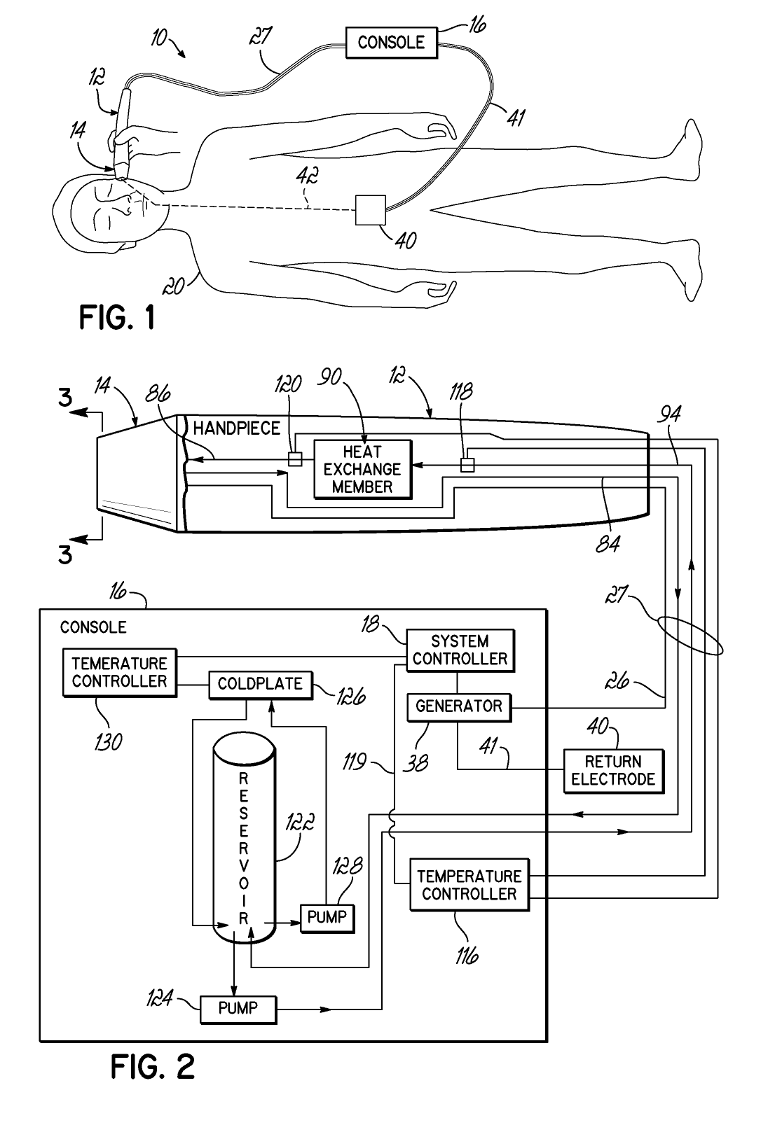

With reference to FIGS. 1-5, a treatment apparatus 10 includes a handpiece 12, a treatment tip 14 coupled in a removable and releasable manner with the handpiece 12, a console generally indicated by reference numeral 16, and a system controller 18. The system controller 18, which is incorporated into the console 16, controls the global operation of the different individual components of the treatment apparatus 10. Under the control of the system controller 18 and an operator's interaction with the system controller 18 at the console 16, the treatment apparatus 10 is adapted to selectively deliver electromagnetic energy in a high frequency band of the electromagnetic spectrum, such as the radiofrequency (RF) band to non-invasively heat a region of a patient's tissue to a targeted temperature range. The elevation in temperature may produce a desired treatment, such as removing or reducing wrinkles and otherwise tightening the skin to thereby improve the appearance of a patient 20 receiving the treatment. In alternative embodiments, the treatment apparatus 10 may be configured to deliver energy in the infrared band, microwave band, or another high frequency band of the electromagnetic spectrum, rather than energy in the RF band, to the patient's tissue.

The treatment tip 14 carries an energy delivery member in the representative form of a treatment electrode 22. The treatment electrode 22 is electrically coupled by conductors inside a cable 27 with a generator 38 configured to generate the electromagnetic energy used in the patient's treatment. In a representative embodiment, the treatment electrode 22 may have the form of a region 26 of an electrical conductor carried on an electrically-insulating substrate 28 composed of a dielectric material. In one embodiment, the substrate 28 may comprise a thin flexible base polymer film carrying the conductor region 26 and thin conductive (e.g., copper) traces or leads 24 on the substrate 28 that electrically couple the conductor region 26 with contact pads 25. The base polymer film may be, for example, polyimide or another material with a relatively high electrical resistivity and a relatively high thermal conductivity. The conductive leads 24 may contain copper or another material with a relatively high electrical conductivity. Instead of the representative solid conductor region 26, the conductor region 26 of treatment electrode 22 may include voids or holes unfilled by the conductor to provide a perforated appearance or, alternatively, may be segmented into plural individual electrodes that can be individually powered by the generator 38.

In one specific embodiment, the treatment electrode 22 may comprise a flex circuit in which the substrate 28 consists of a base polymer film and the conductor region 26 consists of a patterned conductive (i.e., copper) foil laminated to the base polymer film. In another specific embodiment, the treatment electrode 22 may comprise a flex circuit in which the conductor region 26 consists of patterned conductive (i.e., copper) metallization layers directly deposited the base polymer film by, for example, a vacuum deposition technique, such as sputter deposition. In each instance, the base polymer film constituting substrate 28 may be replaced by another non-conductive dielectric material and the conductive metallization layers or foil constituting the conductor region 26 may contain copper. Flex circuits, which are commonly used for flexible and high-density electronic interconnection applications, have a conventional construction understood by a person having ordinary skill in the art.

The substrate 28 includes a contact side 32 that is placed into contact with the skin surface of the patient 20 during treatment and a non-contact side 34 that is opposite to the contact side 32. The conductor region 26 of the treatment electrode 22 is physically carried on non-contact side 34 of the substrate 28. In the representative arrangement, the substrate 28 is interposed between the conductor region 26 and the treated tissue such that, during the non-invasive tissue treatment, electromagnetic energy is transmitted from the conductor region 26 through the thickness of the substrate 28 by capacitively coupling with the tissue of the patient 20.

When the treatment tip 14 is physically engaged with the handpiece 12, the contact pads 25 face toward the handpiece 12 and are electrically coupled with electrical contacts 36, such as pogo pin contacts, inside the handpiece 12. Electrical contacts 36 are electrically coupled with insulated and shielded conductors (not shown) of the electrical wiring 24 also located inside the handpiece 12. The insulated and shielded wires extend exteriorly of the handpiece 12 inside cable 27 to a generator 38 at the console 16. The generator 38, which has the form of a high frequency power supply, is equipped with an electrical circuit (not shown) operative to generate high frequency electrical current, typically in the radio-frequency (RF) region of the electromagnetic spectrum. The operating frequency of generator 38 may advantageously be in the range of several hundred kHz to about twenty (20) MHz to impart a therapeutic effect to treat target tissue beneath a patient's skin surface. The circuit in the generator 38 converts a line voltage into drive signals having an energy content and duty cycle appropriate for the amount of power and the mode of operation that have been selected by the clinician, as understood by a person having ordinary skill in the art.

A non-therapeutic passive or return electrode 40, which is electrically coupled with the generator 38, is physically attached to a site on the body surface of the patient 20, such as the patient's lower back. During treatment, high frequency current flows from the treatment electrode 22 through the treated tissue and the intervening bulk of the patient 20 to the return electrode 40 and then through conductors inside a return cable 41 to define a closed circuit or current path 42. Because of the relatively large surface area of the return electrode 40 in contact with the patient 20, the current density flowing from the patient 20 to the return electrode 40 is relatively low in comparison with the current density flowing from the treatment electrode 22 to the patient 20. As a result, the return electrode 40 is non-therapeutic because negligible heating is produced at its attachment site to the patient 20. High frequency electrical current flowing between the treatment electrode 22 and the patient 20 is maximized at the skin surface and underlying tissue region adjacent to the treatment electrode 22 and, therefore, delivers a therapeutic effect to the tissue region near the treatment site.

As best shown in FIG. 3, the treatment tip 14 includes temperature sensors 44, such as thermistors, that are located on the non-contact side 34 of the substrate 28 that is not in contact with the patient's skin surface. Typically, the temperature sensors 44 are arranged about the perimeter of the conductor region 26 of the treatment electrode 22. Temperature sensors 44 are constructed to detect the temperature of the treatment electrode 22 and/or treatment tip 14, which may be representative of the temperature of the treated tissue. Each of the temperature sensors 44 is electrically coupled by conductive leads 46 with one or more of the contact pads 25, which are used to supply direct current (DC) voltages from the system controller 18 through the electrical wiring 26 to the temperature sensors 44.

With continued reference to FIGS. 1-5, the system controller 18 regulates the power delivered from the generator 38 to the treatment electrode 22 and otherwise controls and supervises the operational parameters of the treatment apparatus 10. The system controller 18 may include user input devices to, for example, adjust the applied voltage level of generator 38. The system controller 18 includes a processor, which may be any suitable conventional microprocessor, microcontroller or digital signal processor, executing software to implement control algorithms for the operation of the generator 38. System controller 18, which may also include a nonvolatile memory (not shown) containing programmed instructions for the processor, may be optionally integrated into the generator 38. System controller 18 may also communicate, for example, with a nonvolatile memory carried by the handpiece 12 or by the treatment tip 14. The system controller 18 also includes circuitry for supplying the DC voltages and circuitry that relates changes in the DC voltages to the temperature detected by the temperature sensors 44.

With specific reference to FIG. 4, the handpiece 12 is constructed from a body 48 and a cover 50 that is assembled with conventional fasteners with the body 48. The assembled handpiece 12 has a smoothly contoured shape suitable for manipulation by a clinician to maneuver the treatment tip 14 and treatment electrode 22 to a location proximate to the skin surface and, typically, in a contacting relationship with the skin surface. An activation button (not shown), which is accessible to the clinician from the exterior of the handpiece 12, is depressed for closing a switch that energizes the treatment electrode 22 and, thereby, delivers high frequency energy over a short delivery cycle to treat the target tissue. Releasing the activation button opens the switch to discontinue the delivery of high frequency energy to the patient's skin surface and underlying tissue. After the treatment of one site is concluded, the handpiece 12 is manipulated to position the treatment tip 14 near a different site on the skin surface for another delivery cycle of high frequency energy delivery to the patient's tissue.

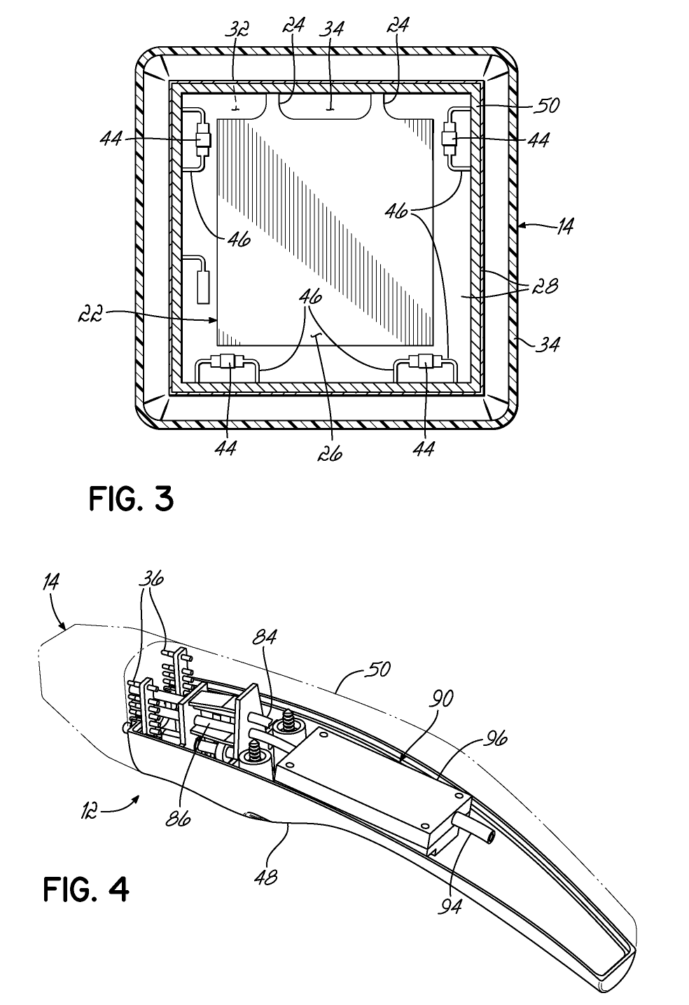

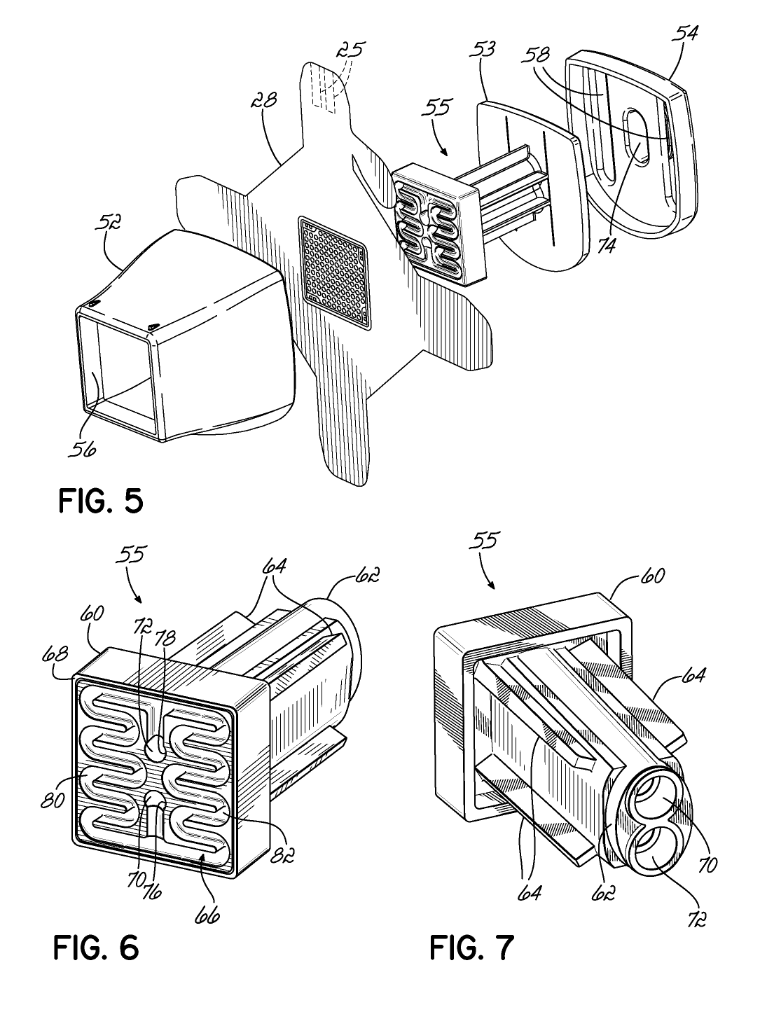

With reference to FIGS. 5-7, the treatment tip 14 includes an outer shell 52, a rear cover 54 that is coupled with an open rearward end of the outer shell 52, a manifold body 55 disposed inside an enclosure or housing inside the outer shell 52, and a flange 53 for the rear cover 54. The flange 53 may be a portion of the manifold body 55. A portion of the substrate 28 overlying the conductor region 26 of the treatment electrode 22 is exposed through a window 56 defined in a forward open end of the outer shell 52. The substrate 28 is wrapped or folded about the manifold body 55. The flange 53 provides a flat support surface over which the contact pads 25 are placed, such that the electrical contacts 36 press firmly against the contact pads 25.

As best shown in FIGS. 5 and 6, the manifold body 55, which may be formed from an injection molded polymer resin, includes a front section 60, a stem 62 projecting rearwardly from the front section 60, and ribs 64 on the stem 62 used to position the manifold body 55 inside the outer shell 52. The front section 60 of the manifold body 55 includes a channel 66 that, in the assembly constituting treatment tip 14, underlines the conductor region 26 of the treatment electrode 22. The shape of the front section 60 corresponds with the shape of the window 56 in the outer shell 52. The substrate 28 of the treatment electrode 22 is bonded with a rim 68 of the manifold body 55 to provide a fluid seal that confines coolant flowing in the channel 66. The area inside the rim 68 is approximately equal to the area of the conductor region 26 of treatment electrode 22. Channel 66 includes convolutions that are configured to optimize the residence time of the coolant in channel 66, which may in turn optimize the heat transfer between the coolant and the treatment electrode 22.

As best shown in FIGS. 5-7, an inlet bore or passage 70 and an outlet bore or passage 72 extend through the stem 62 of the manifold body 55. The inlet passage 70 and outlet passage 72 are rearwardly accessible through an oval-shaped slot 74 defined in the rear cover 54. The inlet passage 70 intersects the channel 66 at an inlet 76 to the channel 66 and the outlet passage 72 intersects the channel 66 at an outlet 78 from the channel 66. The channel 66 is split into two channel sections 80, 82 so that fluid flow in the channel 66 diverges away in two separate streams from the inlet 76 and converges together to flow into the outlet 78. Fluid pressure causes the coolant to flow from the inlet 76 through the two channel sections 80, 82 to the outlet 78 and into the outlet passage 72 and return line 84.

With reference to FIGS. 2 and 5-8, fluid connections are established with the inlet passage 70 and the outlet passages 72 to establish the closed circulation loop and permit coolant flow to the channel 66 in the manifold body 55 when the treatment tip 14 is mated with the handpiece 12. Specifically, the outlet passage 72 is coupled with a return line 84 in the form of a fluid conduit. The inlet passage 70 is coupled by a short conduit or tube 86 with an outlet 88 from a heat exchange member 90, which is physically located inside the handpiece 12 in the representative embodiment. An inlet 92 of heat exchange member 90 is coupled with a supply line 94 in the form of an inlet conduit or tube. The return line 84 and the supply lines 94 extend from the heat exchange member 90 out of the handpiece 12 and are routed to the console 16. The outlet 88 and inlet 92 of the heat exchange member 90, as well as the inlet passage 70 and the outlet passages 72, may include fittings (not shown) that facilitate the establishment of fluid-tight connections.

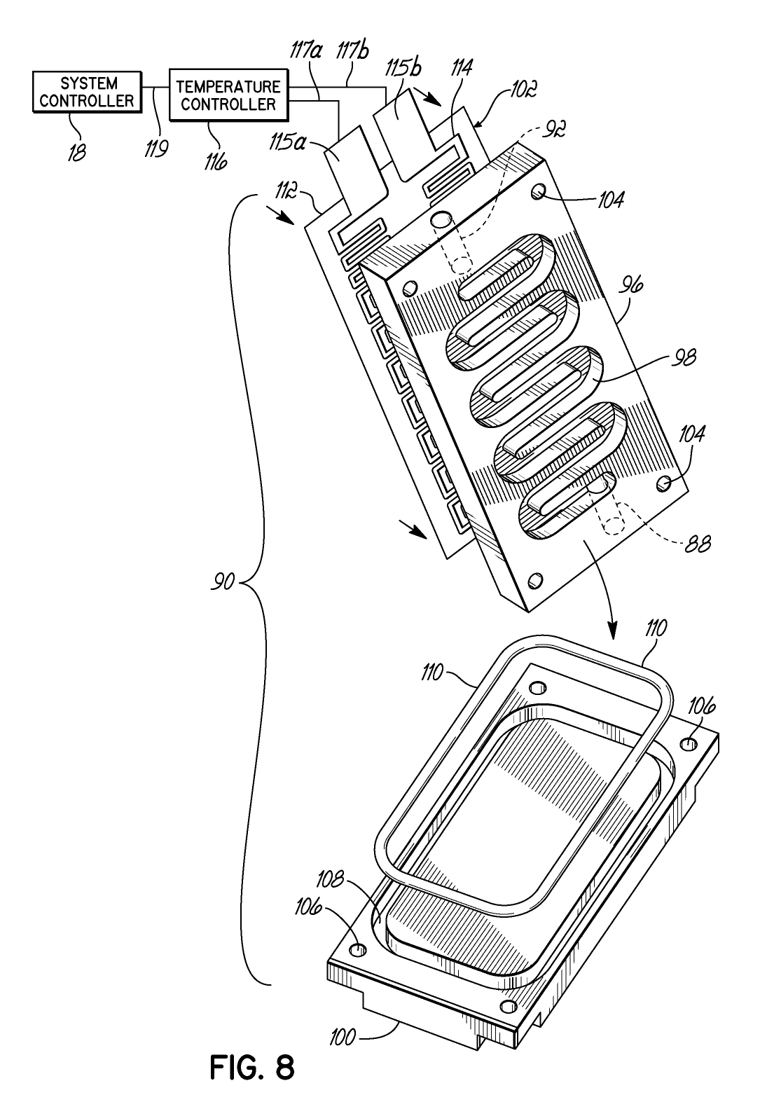

With reference to FIG. 8, the heat exchange member 90 includes a first plate 96, a channel 98 in the first plate 96, a second plate 100, and a heater 102. One end of the channel 98 is coupled with the inlet 92 to the heat exchange member 90 and an opposite end of channel 98 is coupled with the outlet 88 from the heat exchange member 90. The first and second plates 96, 100 include boltholes 104, 106, respectively, positioned near each of the outside corners that are brought into registration when the plates 96, 100 are assembled. The registered boltholes 104, 106 receive fasteners (not shown) used to secure the first and second plates 96, 100 together.

Extending just inside the outer perimeter of the second plate 100 is an o-ring groove 108, which is occupied by a sealing member 110, such as an o-ring. When the first and second plates 96, 100 are secured together using the fasteners, the sealing member 110 is compressed by contact between the first and second plates 96, 100 to an extent sufficient to establish a liquid-tight seal for the channel 98. The first and second plates 96, 100 are formed from a material, such as aluminum, that has a relatively high thermal conductivity to promote efficient heat transfer from the heater 102 to the fluid flowing in the heat exchange member 90.

The heater 102, which is thermally coupled with the first plate 96, includes a substrate 112 of a dielectric material and heating element 114 in the form of a serpentine electrically resistive trace carried on the substrate 112. Opposite ends of the heating element 114 include solder pads 115a, 115b representing external connections that are electrically connected with wiring 117a, 117b leading from the handpiece 12 to a temperature controller 116 located in the console 16. The substrate 112 electrically isolates the heating element 114 from the first plate 96, but permits efficient heat transfer from the heating element 114 to the first plate 96. In one representative embodiment, the heating element 114 may include a flexible polyimide film that measures approximately 1 inch (approximately 2.5 centimeters) by approximately 2 inches (approximately 5.1 centimeters), has an operating voltage of 24 volts, and is adhesively bonded using a layer of a pressure sensitive adhesive to the exterior surface of the first plate 96.

Temperature controller 116 is electrically coupled by a cable 119 for bi-directional communication with system controller 18. The temperature controller 116 includes a power supply that powers the heating element 114. A temperature sensor 118 may be configured to measure the temperature of the coolant in the supply line 94 upstream from the heat exchange member 90. A temperature sensor 120 may be configured to measure the temperature of the coolant in the supply line 94 downstream from the heat exchanger. The temperature sensors 118, 120, which are electrically coupled with the temperature controller 116, are configured to communicate electrical output signals representative of the coolant temperature to the temperature controller 116.

In an alternative embodiment, the temperature sensors 118, 120 may be electrically coupled directly with the system controller 18. In another alternative embodiment, the temperature controller 116 may be consolidated into the system controller 18 to define a single integrated controller. In yet another alternative embodiment, fluid temperatures in the fluid reservoir 122 and in the treatment tip 14 may be utilized to provide the representative coolant temperatures used by the temperature controller 116 to control the heating of the fluid by the heating element 114.

The power delivered to the heating element 114 of heater 102 heats the plates 96, 100 of the heat exchange member 90. Heat energy is transferred from the plates 96, 100 to the coolant flowing in the channel 98, which elevates the output temperature of the coolant at the outlet 88 above its input temperature at the inlet 92. The power delivered to the heating element 114 can be modulated to modify the temperature change of the coolant while the coolant is resident in the channel 98 of the heat exchange member 90. The temperature controller 116 samples the signals from the temperature sensors 118, 120 and supplies output signals representing the temperature difference as feedback to the system controller 18. The system controller 18 determines a desired output temperature for the coolant and provides the output temperature to the temperature controller 116. Based upon the output temperature, the temperature controller 116 adjusts the power supplied to the heating element 114 and, therefore, the amount by which the coolant is heated while flowing through the heat exchange member 90.

The channel 98 has a serpentine path configuration in which the channel 98 includes serpentine convolutions between inlet 92 and outlet 88. Alternatively, the channel 98 can be configured in any non-parallel configuration effective to achieve a similar effect. Specifically, the serpentine path configuration of channel 98 optimizes the residence time of the fluid flowing inside the heat exchange member 90 and maximizes the heat transfer to permit higher fluid flow rates.

With reference to FIG. 2, the treatment apparatus 10 is equipped with a closed loop cooling system that includes the manifold body 55 located inside the treatment tip 14 and the heat exchange member 90 located inside the handpiece 12. The closed loop cooling system further includes a reservoir 122 holding a volume of a coolant and a pump 124, which may be a diaphragm pump, that continuously pumps a stream of the coolant from an outlet of the reservoir 122 through the supply line 94 to the heat exchange member 90. The manifold body 55 is coupled in fluid communication with the reservoir 122 by the return line 84. The return line 84 conveys the coolant from the treatment tip 14 and handpiece 12 back to the reservoir 122 to complete the circulation loop.

Heat generated in the treatment tip 14 by energy delivery from the treatment electrode 22 and heat transferred from the patient's skin and an underlying depth of heated tissue is conducted through the substrate 28 and treatment electrode 22. The heat is absorbed by the circulating coolant in the channel 66 of the manifold body 55, which lowers the temperature of the treatment electrode 22 and substrate 28 and, thereby, cools the patient's skin and the underlying depth of heated tissue. The cooling, at the least, assists in regulating the depth over which the tissue is heated to a therapeutic temperature by the delivered electromagnetic energy.

The coolant is chilled by a separate circulation loop 125 that pumps coolant from the reservoir 122 through separate supply and return lines to a coldplate 126. A pump 128, which may be a centrifugal pump, pumps the coolant under pressure from the reservoir 122 to the coldplate 126. In an alternative embodiment, the coldplate 126 may be placed directly in the return line 84 if permitted by the capacity of the coldplate 126 and flow constrictions.

In a representative embodiment, the coldplate 126 may be a liquid-to-air heat exchanger that includes a liquid heat sink with a channel (not shown) for circulating the coolant, a thermoelectric module (not shown), and an air heat sink (not shown). A cold side of the thermoelectric module in coldplate 126 is thermally coupled with the liquid heat sink and a hot side of the thermoelectric module in coldplate 126 is thermally coupled with the air heat sink. The cold side is cooled for extracting heat from the coolant flowing through the liquid heat sink. As understood by a person having ordinary skill in the art, an array of semiconductor couples in the thermoelectric module operate, when biased, by the Peltier effect to convert electrical energy into heat pumping energy. Heat flows from the liquid heat sink through the thermoelectric elements to the air heat sink. The air heat sink of the liquid-to-air heat exchanger dissipates the heat extracted from the coolant circulating in the liquid heat sink to the surrounding environment. The air heat sink may be any conventional structure, such as a fin stack with a fan promoting convective cooling.

A temperature controller 130 inside the console 16 is electrically coupled with the coldplate 126 and is also electrically coupled with the system controller 18. The system controller 18, which is electrically coupled with a temperature sensor (not shown) used to measure the coolant temperature in the reservoir 122, supplies command signals to the temperature controller 130 in response to the measured coolant temperature. Under the feedback control, the temperature of the coolant in the reservoir 122 is regulated by controlling the operation of the coldplate 126.

In use and with reference to FIGS. 1-8, the coolant is circulated by pump 128 between the coldplate 126 and the reservoir 122. The system controller 18 monitors the temperature of the coolant in the reservoir 122 and communicates with the temperature controller 130 to establish a temperature for the coolant in the reservoir 122. The system controller 18 samples electrical signals communicated from the reservoir temperature sensor for use in setting the coolant temperature in the reservoir 122. The coolant temperature is established in the reservoir 122 at a calculated temperature setting that is less than the minimum desired temperature at the treatment tip 14. In other words, the coolant temperature in the reservoir 122 is set at a value that is colder than the coolant temperature required at the treatment tip 14.

The over-cooling is necessary as the coolant will inevitably warm as it passes through supply line 94 from the console 16 to the handpiece 12. This warming can be minimized by insulating the exterior of the supply line 94 to limit heat gain from the environment, but cannot be eliminated. Further complicating the problem, the amount of heat transferred to the coolant will vary based on the ambient room temperature and fluid flow rate. By cooling the coolant to a temperature lower than desired, then warming in the handpiece 12 just prior to delivery to the treatment tip 14, coolant can be delivered to the treatment tip 14 at the desired temperature at much greater accuracy than without this process.

The coolant is continuously pumped by pump 124 through the supply line 94 from the reservoir 122 to the handpiece 12. The system controller 18 relies on the upstream and downstream temperatures measured by the temperature sensors 118, 120 to regulate the power supplied to the heating element 114. Based upon the output signals from the temperature sensors 118, 120, the system controller 18 calculates a temperature differential of the coolant upstream and downstream of the heat exchange member 90. The system controller 18 communicates control signals to the temperature controller 116 based upon the temperature differential. The temperature controller 116 translates the control signals into a power level for the heating element 114 of heater 102, which powers the heating element 114 to heat the heat exchange member 90. The temperature of the coolant is elevated by heat transferred from the heat exchange member 90 to a desired temperature before delivery to the treatment tip 14. Because the heating is occurring locally in the handpiece 12 and based upon measured temperatures of the coolant in the handpiece 12, the coolant temperature can be accurately regulated.

The coolant, which is at the desired temperature, is delivered to the manifold body 55 and circulated through the channel 66 in contact with the conductor region 26 of treatment electrode 22 on the non-contact side 34 of substrate 28. This cools the treatment electrode 22, which in turn cools the tissue immediately beneath the patient's skin surface in the contacting relationship with the contact side 32 of the substrate 28. Spent coolant is directed from the channel 66 into the return line 84 and returned to the reservoir 122.

The treatment electrode 22 is energized by generator 38 to deliver high frequency energy to the target tissue. The continuous stream of coolant flowing through the channel 66 in the manifold body 55 continuously cools the adjacent tissue contacted by the treatment electrode 22. The cooling prevents superficial tissue from being heated to a temperature sufficient to cause a significant and possibly damaging thermal effect. Depths of tissue that are not significantly cooled by thermal energy transfer to the continuous stream of coolant flowing through the channel 66 in manifold body 55 will be warmed by the high frequency energy to therapeutic temperatures resulting in the desired therapeutic effect. The amount or duration of pre-cooling, after the treatment electrode 22 is contacted with the skin surface and before electromagnetic energy is delivered, may be used to select the protected depth of untreated tissue. Longer durations of pre-cooling and lower coolant temperatures produce a deeper protected zone and, hence, a deeper level in tissue for the onset of the treatment zone.

Using the same mechanism, the tissue is also cooled by the continuous stream of coolant flowing through the manifold body 55 during energy delivery and after heating by the transferred high frequency energy. Post-cooling may prevent or reduce heat delivered deeper into the tissue from conducting upward and heating shallower depths to therapeutic temperatures even though external energy delivery from the treatment electrode 22 to the targeted tissue has ceased.

While the invention has been illustrated by a description of various embodiments and while these embodiments have been described in considerable detail, it is not the intention of the applicant to restrict or in any way limit the scope of the appended claims to such detail. Additional advantages and modifications will readily appear to those skilled in the art. Thus, the invention in its broader aspects is therefore not limited to the specific details, representative apparatus and method, and illustrative example shown and described. Accordingly, departures may be made from such details without departing from the spirit or scope of applicant's general inventive concept.

What is claimed is:

1. An apparatus for treating tissue with electromagnetic energy, the apparatus comprising:
an energy delivery device configured to transfer the electromagnetic energy to the tissue, the energy delivery device including a manifold body, a channel in the manifold body, an inlet to the channel, and an outlet from the channel;
a closed-loop cooling system coupled in a first circulation loop with the inlet and the outlet of the channel, the closed-loop cooling system including a first pump configured to pump a fluid in the first circulation loop to the inlet of the channel and through the channel to the outlet from the channel; and
a heat exchange member disposed in the first circulation loop between the first pump and the inlet to the channel, the heat exchange member configured to heat the fluid before the fluid enters the inlet of the channel.

2. The apparatus of claim 1 wherein the energy delivery device is a treatment tip, and further comprising:
a handpiece configured to receive the treatment tip and to establish a fluid connection with the channel in the manifold body.

3. The apparatus of claim 2 wherein the heat exchange member is disposed in the handpiece.

4. The apparatus of claim 2 wherein the treatment tip further includes a treatment electrode configured to transfer the electromagnetic energy to the tissue, the treatment electrode being positioned within the treatment tip to conduct heat from the tissue to the fluid in the channel of the manifold body.

5. The apparatus of claim 4 wherein the treatment tip further includes a dielectric substrate disposed between the treatment electrode and a skin surface overlying the tissue such that, during a non-invasive tissue treatment, the electromagnetic energy is transmitted from the treatment electrode through the dielectric substrate by capacitive coupling with the tissue.

6. The apparatus of claim 1 wherein the closed-loop cooling system includes a reservoir holding the fluid, and further comprising:
a coldplate configured to cool the fluid, the coldplate coupled in a second circulation loop with the reservoir.

7. The apparatus of claim 6 further comprising:
a second pump configured to pump the fluid through the second circulation loop.

8. The apparatus of claim 7 wherein the coldplate includes a thermoelectric module and a liquid heat sink in thermal contact with the thermoelectric module, the liquid heat sink including a flow path coupled with the reservoir, and the thermoelectric module having a cold side thermally coupled with the liquid heat sink.

9. The apparatus of claim 1 wherein the heat exchange member includes a heating element configured to transfer heat to the fluid, and further comprising:
a temperature controller electrically coupled with the heating element, the temperature controller configured to power the heating element to heat the fluid in the channel.

10. The apparatus of claim 9 further comprising:
a first temperature sensor configured to measure a first temperature of the fluid in the first circulation loop at a first location between the pump and the heat exchange member, the first temperature sensor electrically coupled with the temperature controller for providing output signals reflecting the first temperature; and
a second temperature sensor configured to measure a second temperature of the fluid in the first circulation loop at a second location between the heat exchange member and the manifold body, the second temperature sensor electrically coupled with the temperature controller for providing output signals reflecting the second temperature.

11. The apparatus of claim 10 wherein the temperature controller is configured to determine a temperature difference between the first temperature and the second temperature, and further comprising:
a system controller electrically coupled with the temperature controller, the system controller configured to determine an output temperature for the fluid based upon the temperature difference received from the temperature controller and to communicate the output temperature to the temperature controller for use in powering the heating element to heat the fluid to the output temperature.

12. The apparatus of claim 1 wherein the heat exchange member includes a first plate, a second plate, a flow passage between the first plate and the second plate, and a heating element coupled with at least one of the first plate or the second plate for transferring thermal energy to the fluid flowing in the flow passage.

13. A method for treating tissue beneath a skin surface with electromagnetic energy, the method comprising:
pumping a fluid from a reservoir to an energy delivery device;
heating the fluid at a location between the reservoir and the energy delivery device;
after the fluid is heated, circulating the fluid through the energy delivery device;
returning the fluid from the energy delivery device to the reservoir; and
delivering the electromagnetic energy from the energy delivery device to the tissue.

14. The method of claim 13 further comprising:
cooling the fluid in the reservoir to a fluid temperature less than an ambient temperature.

15. The method of claim 14 wherein cooling the fluid further comprises:
circulating the fluid from the reservoir to a coldplate configured to cool the fluid sufficiently to maintain the fluid in the reservoir at the fluid temperature.

16. The method of claim 14 wherein the delivery of the electromagnetic energy heats the tissue, and heating the fluid further comprises:
transferring heat energy from an outer layer of the tissue near the skin surface to the fluid circulating through the energy delivery device; and
heating the fluid to a different fluid temperature suitable to regulate the transfer of the heat energy.

17. The method of claim 16 wherein heating the fluid to the different temperature controls a depth from the skin surface into the tissue from which heat energy is transferred.

18. The method of claim 13 further comprising:
contacting the skin surface with a portion of the energy delivery device while delivering the electromagnetic energy to the tissue.

19. The method of claim 13 further comprising:
contacting a skin surface with a portion of the energy delivery device while delivering the electromagnetic energy to the tissue in a non-invasive manner.

20. The method of claim 13 wherein delivering the electromagnetic energy further comprises:
capacitively coupling the electromagnetic energy from the energy delivery device to the tissue beneath the skin surface.

21. The method of claim 13 wherein heating the fluid further comprises:
transferring thermal energy from a heat exchange member to the fluid.

22. The method of claim 21 wherein the heat exchange member is located in a handpiece, and the energy delivery device is carried in a treatment tip coupled with the handpiece.

23. The method of claim 21 wherein heating the fluid further comprises:
measuring a temperature difference between the fluid before entering an inlet to the heat exchange member and the fluid after exiting an outlet from the heat exchange member; and
delivering the thermal energy from the heat exchange member to the fluid at a rate determined by the temperature difference.

24. The method of claim 13 wherein delivering the electromagnetic energy further comprises:
delivering the electromagnetic energy from a treatment electrode of the energy delivery device to the tissue beneath the skin surface so as to heat the tissue.

25. The method of claim 24 further comprising:
contacting the skin surface with a portion of the energy delivery device while delivering the electromagnetic energy from the treatment electrode to the tissue; and
transferring heat energy from an outer layer of the tissue near the skin surface through the treatment electrode to the fluid circulating through the energy delivery device.

26. The method of claim 25 wherein circulating the fluid further comprises:
circulating the fluid in contact with the treatment electrode.
